(19) United States
(12) Patent Application Publication
(10) Pub. No.: US 2026/0182934 A1
(43) Pub. Date: Jul. 2, 2026

(54) DYNAMIC FILTER FOR RADIOGRAPHY SYSTEM

(71) Applicant: UNIVERSITY OF PITTSBURGH-OF THE COMMONWEALTH SYSTEM OF HIGHER EDUCATION, Pittsburgh, PA (US)

(72) Inventors: William Anderst, Pittsburgh, PA (US); Tom Gale, Pittsburgh, PA (US)

(73) Assignee: UNIVERSITY OF PITTSBURGH-OF THE COMMONWEALTH SYSTEM OF HIGHER EDUCATION, Pittsburgh, PA (US)

(21) Appl. No.: 19/131,678

(22) PCT Filed: Dec. 11, 2023
(86) PCT No.: PCT/US2023/083332

§ 371 Date: May 21, 2025

Related U.S. Application Data
(60) Provisional application No. 63/387,751, filed on Dec. 16, 2022.

(51) Int. Cl. A61B 6/02; A61B 6/00; A61B 6/06; A61B 6/40

(52) U.S. Cl. CPC A61B 6/022; A61B 6/06; A61B 6/4014; A61B 6/4035; A61B 6/54

(57) ABSTRACT

A dynamic filter for a radiographic imaging system includes a frame having an interface member structured for coupling the dynamic filter to an x-ray source of the radiographic imaging system, a movable member made of an x-ray blocking material that is structured to block a portion of an incident x-ray beam of the x-ray source to prevent it from travelling to an x-ray detector of the radiographic imaging system, and a motor supported by the frame and coupled to the movable member, wherein the motor is structured and configured to control operation of the motor to move moveable member based on data indicative of a position of and/or movement of a patient

### CROSS REFERENCE TO RELATED APPLICATIONS

This application claims priority to U.S. Provisional Patent Application Ser. No. 63/387,751, filed on Dec. 16, 2022 and titled “Dynamic Filter for Radiography System,” the disclosure of which is incorporated herein by reference.

### STATEMENT OF GOVERNMENT INTEREST

This invention was made with government support under grant #AR076725 awarded by the National Institutes of Health (NIH). The government has certain rights in the invention.

### FIELD OF THE INVENTION

The present invention pertains to radiography systems, such as dynamic single-plane radiography systems or dynamic stereo radiography systems, and, in particular, to a radiography system having a dynamic filter for reducing the amount of washout in images captured by the system.

### BACKGROUND OF THE INVENTION

Low back disorders are one of the most significant causes of years lived with disability worldwide, ranking first amongst musculoskeletal disorders. Although low back pain is complex and multi-factorial, identification of abnormal kinematics is an accepted basis for clinical decision-making. However, current kinematics-based metrics for diagnosis of lower back disorders are based primarily on static imaging modalities such as lateral X-ray images or supine MRI. At best, these modalities provide linear, scalar, or discrete values, such as end-range of motion and fixed center of rotation. While useful, they remain inadequate to comprehensively capture nonlinear kinematic waveform patterns that occur during everyday movements and may differentiate between healthy and symptomatic cohorts.

In spite of numerous studies investigating how the human lumbar spine moves, three-dimensional (3D) in vivo kinematic data for intervertebral joints is sparse, limiting the understanding of what constitutes “normal lumbar kinematics.” Current in vivo data acquisition techniques are unable to quantify 3D lumbar vertebral kinematics with sufficient accuracy. End-range of motion 2D functional flexion-extension radiographs, presently the standard diagnostic tool, miss at least four important characteristics of lumbar spinal motion: (a) midrange motion characteristics, (b) out-of-plane or coupled motion patterns, (c) effects of dynamic muscle forces and external loading on individual vertebral motion paths, and (d) potential nonlinear relationships between instantaneous vertebral motion and overall trunk motion. The last two limitations necessarily extend to static studies utilizing dual plane X-ray imaging systems, although such studies could provide 3D information regarding out-of-plane motion patterns. Similar limitations apply to MRI- and CT-based approaches, wherein subjects are generally in a supine, non-weight-bearing position, and thus in a nonfunctional loading state. Surface marker-based motion analysis can provide dynamic 3D data, but is prone to significant skin motion artifacts and inaccurate identification of specific bony landmarks by palpation, which hamper the ability to accurately estimate underlying vertebral motion. The putative reduction in error by using bone pins cannot be justified for most studies given their extreme invasiveness.

Continuous X-ray imaging techniques can potentially overcome drawbacks of traditional measurement techniques, since dynamic bone motion can be directly recorded in vivo without a surgically invasive procedure. Several studies have demonstrated the ability to capture continuous spinal segmental motion in the sagittal plane using cineradiography or digital fluoroscopy video (DFV). However, all of these studies employed single-plane X-ray imaging, precluding acquisition of out-of-plane or coupled motion patterns. Furthermore, studies relying on manual identification of anatomical landmarks in X-ray images are vulnerable to reduced accuracy. Inherent limitations of DFV hardware with respect to maximum frame rate (<30 fps) and minimum exposure time of pulsed radiation (>8 ms) either blur the dynamically acquired images when the movement of interest is too fast or restrict the study to movements performed at very slow speeds.

An ideal technique should have the capability to directly record continuous 3D vertebral motion at a desired speed in a minimally invasive manner without altering the posture of the subject performing functional tasks. It should subsequently track the bone motion with submillimeter accuracy. Such data are now available for other joints like the knee, shoulder, and cervical spine. However, obtaining dynamic 3D data for lumbar joints continues to be challenging, given the relatively complex anatomy and voluminous soft tissue content compared to other anatomical regions. The ability to comprehensively and accurately examine 3D dynamic function of the lumbar spine could lead to advancements in areas such as: (a) clinical evaluation of lower back disorders, (b) biomechanical model-based predictions of disk forces and stresses during functional tasks, and (c) design of disk replacements to more closely replicate the natural biomechanics of the lumbar spine. Availability of accurate, continuous in vivo intervertebral kinematics for specific functional tasks can particularly contribute to improving the accuracy of lumbar biomechanical models. Currently, forces occurring in the disk and facet joints cannot be measured in vivo without extremely invasive procedures. Instead, such forces are inferred or estimated from biomechanical models. However, biomechanical models are sensitive to the accuracy of kinematic input. Improving the accuracy of kinematic input into lumbar biomechanical models is a critical prerequisite for attaining better insight into forces, moments, and stresses acting on the joints in vivo.

Another problem that exists in this field is what is known as radiation whiteout. Radiation whiteout refers to an overexposure of the X-ray image intensifier due to large areas of unattenuated radiation, causing a “washing out” of the images. For example, images acquired from the medial-lateral (ML) direction during an flexion-extension movement will typically “wash out” as the participant moves from an upright to a flexed position.

### SUMMARY OF THE INVENTION

In one embodiment, a radiographic imaging system is provided that includes a position and motion detecting apparatus structured and configured to be worn by a patient and to generate data indicative of a position of and/or movement of the patient, an x-ray source structured to generate an incident x-ray beam, an x-ray detector structured to detect a transmitted x-ray beam, and a dynamic filter coupled to the x-ray source. The dynamic filter has a movable member made of an x-ray blocking material and is structured to block a portion of the incident x-ray beam to prevent it from travelling to the x-ray detector. A controller is coupled to the position and motion detecting apparatus and the dynamic filter, wherein movement and positioning of the movable member is controlled by the controller based on the data indicative of position of and/or movement of the patient.

In another embodiment, a dynamic filter for a radiographic imaging system is provided. The dynamic filter includes a frame having an interface member structured for coupling the dynamic filter to an x-ray source of the radiographic imaging system, a movable member made of an x-ray blocking material that is structured to block a portion of an incident x-ray beam of the x-ray source to prevent it from travelling to an x-ray detector of the radiographic imaging system, and a motor supported by the frame and coupled to the movable member, wherein the motor is structured and configured to control operation of the motor to move moveable member based on data indicative of a position of and/or movement of a patient.

In still another embodiment, a radiographic imaging method is provided that includes generating an incident x-ray beam, generating data indicative of a position of and/or movement of the patient during a movement task, and controlling movement and positioning of a movable member of a dynamic filter based on the data indicative of position of and/or movement of the patient to block a portion of the incident x-ray beam to prevent it from travelling to an x-ray detector.

### BRIEF DESCRIPTION OF THE DRAWINGS

A full understanding of the invention can be gained from the following description of the preferred embodiments when read in conjunction with the accompanying drawings in which:

FIGS. 1 and 2 are schematic diagrams of a dynamic stereo radiography system according to an exemplary embodiment of the disclosed concept;

FIG. 3 is an isometric view of a dynamic filter forming part of the dynamic stereo radiography system of FIGS. 1 and 2 according to one non-limiting, exemplary embodiment of the disclosed concept; and

FIG. 4 is an exploded view of the dynamic filter of FIG. 3.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the term “inertial measurement unit (IMU)” shall mean a position and motion detecting apparatus that employs multiple sensors for measuring orientation, angular rate, and/or acceleration/forces by combining one or more accelerometers, one or more gyroscopes, and one or more magnetometers into one apparatus.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details to provide a thorough understanding of the subject invention. It will be evident, however, that the disclosed concept can be practiced without these specific details without departing from the spirit and scope of this innovation.

As described in detail herein in connection with various exemplary embodiments, the disclosed concept provides a dynamic filter for a dynamic stereo radiography system, and a dynamic stereo radiography system employing same, that minimizes the amount of radiation wash-out occurring during image capture. Wash-out is minimized because the dynamic filter of the disclosed concept is able to dynamically block certain radiation during the imaging process based on patient position as described herein. The disclosed concept thus provides an improved dynamic stereo radiography system that enables the 3D reconstruction of shape, position, and orientation of the vertebrae in a patient's spine. Specifically, a three-dimensional reconstruction of the movement of the spine, with minimized wash-out effects, may be generated based on a series of multi-frame radiographic (i.e., x-ray) images of the patient's spine acquired using the dynamic stereo radiography system of the disclosed concept.

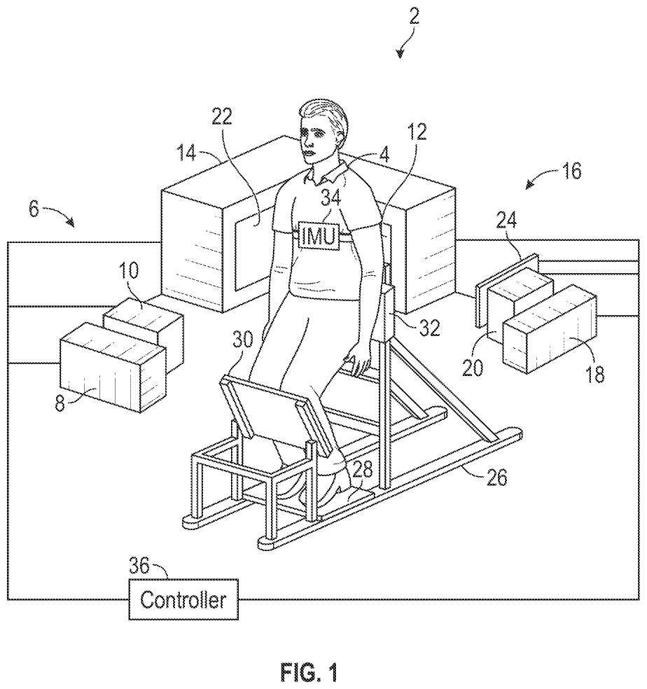

FIGS. 1 and 2 are schematic diagrams of a dynamic stereo radiography system 2 according to an exemplary embodiment of the disclosed concept. As described in detail herein, dynamic stereo radiography system 2 is an imaging system that enables the 3-D reconstruction of shape, position, and orientation of the spine of a patient 4. While the disclosed concept is described for illustrative purposes herein in connection with dynamic stereo radiography system 2, it will be understood that other radiographic imaging systems, such as single-plane radiographic imaging systems, may also be employed in connection with implementation of the disclosed concept.

Dynamic stereo radiography system 2 includes a first x-ray imaging system 6 including an x-ray source 8, a collimator 10, and an x-ray detector panel 12 that is provided within a reference box 14. Dynamic stereo radiography system 2 further includes a second x-ray imaging system 16 including an x-ray source 18, a collimator 20, a dynamic filter 24 (described in more detail below), and an x-ray detector panel 22 provided within reference box 14. As seen in FIGS. 1 and 2, first x-ray imaging system 6 and second x-ray imaging system 16 are positioned at an angle with respect to one another such that the x-ray beams 37, 38 thereof overlap in part to create a 3-D viewing volume 40. In the non-limiting illustrated embodiment, first x-ray imaging system 6 is configured in the anterior-posterior (AP) direction, and second x-ray imaging system 16 is configured in the medial-lateral (ML) direction, although it will be appreciated that other configurations are also contemplated within the scope of the disclosed concept.

In addition, as noted above, second x-ray imaging system 16 further includes dynamic filter 24 that is coupled to collimator 20. Also, as seen in FIG. 1, dynamic stereo radiography system 2 further includes an inertial measurement unit (IMU) 34 that is structured to be worn by patient 4 during the imaging process as described herein. IMU 34 is structured and configured to measure a number of position and/or motion parameters of patient 4 during operation of dynamic stereo radiography system 2. As noted elsewhere herein, in the illustrated embodiment employing IMU 34, the position and motion parameters include orientation, angular rate, and acceleration/force parameters. Dynamic filter 24 and IMU 34 together work to automatically and dynamically block certain areas of radiation according to the position of patient 4 during use of dynamic stereo radiography system 2. In this manner, the amount of wash-out occurring during operation of dynamic stereo radiography system 2 will be minimized. While the exemplary embodiment employs IMU 34 as the position and motion detecting apparatus, it will be understood, however, that IMU 34 is meant to be exemplary only, and that other position and motion detecting apparatuses may also be employed within the scope of the disclosed concept.

Furthermore, dynamic stereo radiography system 2 includes a support structure 26 for supporting patient 4 during the imaging process. As seen in FIG. 1, support structure 24 includes a foot support portion 28 a knee support portion 30, and a pelvic support portion 32.

Dynamic stereo radiography system 2 still further includes a controller 36 that is operatively coupled to the operational components of dynamic stereo radiography system 2 as seen in FIGS. 1 and 2. Controller 36 stores a number of software instructions/routines for controlling operation of dynamic stereo radiography system 2 as described herein, including the automatic and dynamic control of dynamic filter 24 based on the output of IMU 34. While one controller 36 is described in connection with the exemplary embodiment, it will be understood that the functionality described herein may be spread over multiple individual controlling devices. For example, the control of dynamic stereo radiography system 2 may be handled in a controlling device that is separate from the controlling device that handles the control of dynamic filter 24 IMU 34.

In operation, the target region of the spine of patient 4 is positioned and maintained within 3-D viewing volume 40 throughout a series of exposures/image captures of patient 4 with x-ray imaging systems 6 and 16 while patient 4 is executing a certain, predetermined given range of motion task. In the exemplary embodiment, the range of motion task performed by patient 4 comprises a lifting task wherein patient 4 bends over and lifts an object of a known weight from a starting, trunk flexed position to a final, upright position in a sagittally symmetric manner. As a result, a dynamic, multi-frame series of images is captured by dynamic stereo radiography system 2 in order to enable the 3-D reconstruction of shape, position, and orientation of the spine of a patient 4. In addition, as patient 4 moves, information indicative of the position and/or movement of patient 4 is detected by IMU 34 and is provided to controller 36. In response, controller 36 controls operation of dynamic filter 24 so as to dynamically block (partially) the radiation from x-ray source 18 and collimator 20 according to patient position as measured by IMU 34. As a result of this dynamic blocking of radiation, the amount of wash-out that will occur during the imaging process will be minimized, as only a portion of beam 38 will be allowed to travel to detector 22.

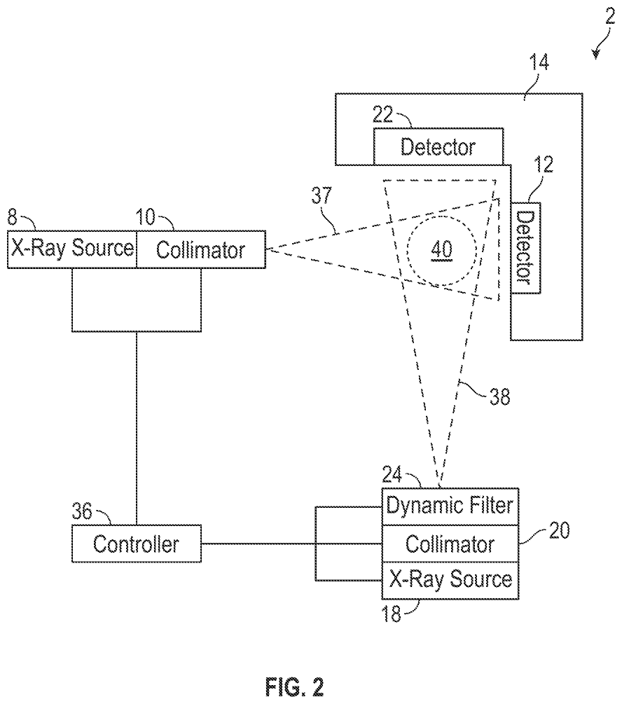

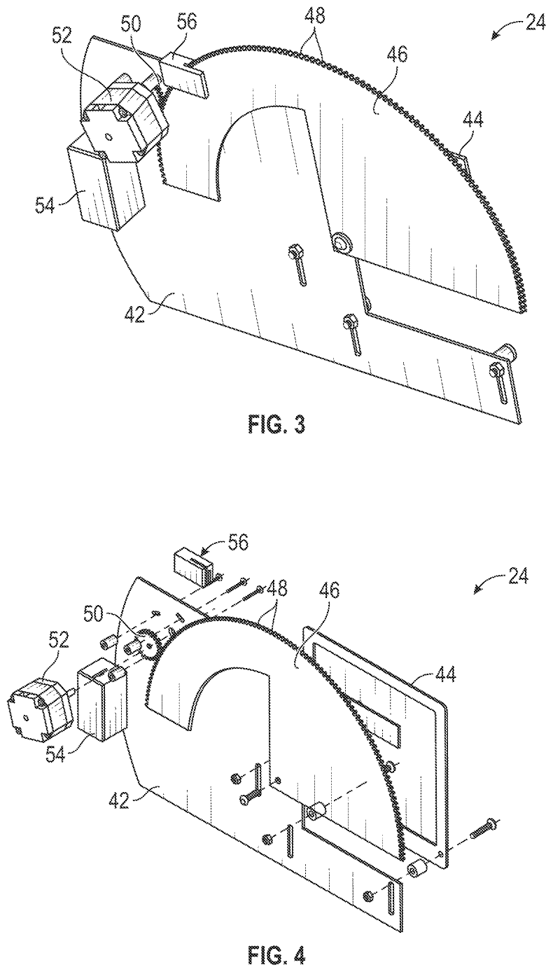

FIG. 3 is an isometric view of dynamic filter 24 according to one non-limiting, exemplary embodiment of the disclosed concept. FIG. 4 is an exploded view of dynamic filter 24 according to this non-limiting exemplary embodiment. Dynamic filter 24 includes a main frame 42 structured to hold the components of dynamic filter 24. Dynamic filter 24 also includes an interface member 44 that is structured to couple dynamic filter 24 to collimator 20. In the exemplary embodiment, interface member is adjustable in the vertical direction. A semicircular blade 46 having a plurality of teeth 48 is held in front of main frame 42. Blade 46 is structured and configured to dynamically block radiation from x-ray source 18 and collimator 20 according to patient position as measured by IMU 34. In the exemplary embodiment, blade 46 is made of stainless steel layered with lead.

A spur gear 50 is held by main frame 42. The teeth of spur gear 50 are mated with teeth 48 of blade 46 so that spur gear 50 is able to drive rotational movement of blade 46 about a central hinge thereof. Main frame 42 also supports a stepper motor 52 and a motor connector 54. Stepper motor 52 is coupled to and drives spur gear 50 under the control of controller 36. Motor connector 54 houses the connector for connecting stepper motor 52 to controller 36. A slotted blade guide 56 is held by main frame 42. Blade guide 56 provides stability for blade 46 as it is moved as described herein.

In operation, as patient 4 moves to perform the range of motion task, first x-ray imaging system 6 and second x-ray imaging system 16 operate simultaneously to capture the images needed to enable the 3-D reconstruction. At the same time, as patient moves 4, IMU 34 generates data relating to the orientation, angular rate, and acceleration/forces of patient 4. That data is provided to controller 36. In turn, based on that data, controller 36 controls operation of stepper motor 52 in order to move blade 46 into the appropriate position. As patient 4 moves and as blade 46 is moved accordingly, excess radiation from collimator 20 will be blocked by blade 46, resulting in a reduction in the amount of wash-out that occurs. In the non-limiting exemplary embodiment, the data gathered by IMU 34 is converted into a single orientation angle (sagittal plane trunk flexion angle) per clock cycle. This trunk flexion angle (the data indicative of a position of and/or movement of patient 4) is fed to controller 36. The controller then drives spur gear 50 a set amount of steps to match the angle of the edge of blade 46 to the trunk angle (blade edge upright and person standing upright are considered 0 degrees).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A radiographic imaging system, comprising:
a position and motion detecting apparatus structured and configured to be worn by a patient and to generate data indicative of a position of and/or movement of the patient; an x-ray source structured to generate an incident x-ray beam;
an x-ray detector structured to detect a transmitted x-ray beam;
a dynamic filter coupled to the x-ray source, the dynamic filter having a movable member made of an x-ray blocking material and structured to block a portion of the incident x-ray beam to prevent it from travelling to the x-ray detector; and
a controller coupled to the position and motion detecting apparatus and the dynamic filter, wherein movement and positioning of the movable member is controlled by the controller based on the data indicative of position of and/or movement of the patient.

2. The radiographic imaging system according to claim 1, wherein the radiographic imaging system is a dynamic stereo radiographic imaging system having the x-ray source and the x-ray detector, and a second x-ray source and second x-ray detector, wherein the x-ray source and x-ray detector are configured in the medial-lateral (ML), and the second x-ray source and second x-ray detector are configured in the direction anterior-posterior (AP) direction.

3. The radiographic imaging system according to claim 1, further comprising a collimator coupled to the x-ray source, wherein the dynamic filter is directly coupled to the collimator.

4. The radiographic imaging system according to claim 1, wherein the dynamic filter includes a frame supporting the movable member, and a motor coupled to the movable member, wherein the motor controls operation of the motor to move the moveable member based on the data indicative of position of and/or movement of the patient.

5. The radiographic imaging system according to claim 1, wherein the movable member comprises a blade member.

6. The radiographic imaging system according to claim 5, wherein the dynamic filter includes a gear, wherein the blade member is coupled to the gear, and wherein the gear is driven by the motor under control of the controller.

7. The radiographic imaging system according to claim 6, wherein the dynamic filter includes a slotted blade guide, wherein the blade member is movably received within a slot of the slotted blade guide.

8. The radiographic imaging system according to claim 5, wherein the blade member is semicircular blade rotatably held by the frame.

9. The radiographic imaging system according to claim 1, wherein the position and motion detecting apparatus is an inertial measurement unit.

10. A dynamic filter for a radiographic imaging system, comprising:
a frame having an interface member structured for coupling the dynamic filter to an x-ray source of the radiographic imaging system;
a movable member made of an x-ray blocking material and being structured to block a portion of an incident x-ray beam of the x-ray source to prevent it from travelling to an x-ray detector of the radiographic imaging system; and
a motor supported by the frame and coupled to the movable member, wherein the motor is structured and configured to control operation of the motor to move moveable member based on data indicative of a position of and/or movement of a patient.

11. The dynamic filter according to claim 10, wherein the interface member is structured for directly coupling the dynamic filter to a collimator coupled to the x-ray source.

12. The dynamic filter according to claim 10, wherein the movable member comprises a blade member.

13. The dynamic filter according to claim 12, further comprising a gear, wherein the blade member is coupled to the gear, and wherein the gear is driven by the motor.

14. The dynamic filter according to claim 13, further comprising a slotted blade guide, wherein the blade member is movably received within a slot of the slotted blade guide.

15. The dynamic filter according to claim 12, wherein the blade member is semicircular blade rotatably held by the frame.

16. The dynamic filter according to claim 10, wherein the data indicative of a position of and/or movement of the patient is generated by an inertial measurement unit.

17. A radiographic imaging method, comprising:
generating an incident x-ray beam;
generating data indicative of a position of and/or movement of the patient during a movement task; and
controlling movement and positioning of a movable member of a dynamic filter based on the data indicative of position of and/or movement of the patient to block a portion of the incident x-ray beam to prevent it from travelling to an x-ray detector.

18. The radiographic imaging method according to claim 17, wherein the generating of the data is performed by an inertial measurement unit.

19. The radiographic imaging method according to claim 17, wherein the dynamic filter includes a frame supporting the movable member, and a motor coupled to the movable member, wherein the motor controls operation of the motor to move the moveable member based on the data indicative of position of and/or movement of the patient.

20. The radiographic imaging method according to claim 17, wherein the movable member comprises a semicircular blade member.
